(19) United States
(12) Patent Application Publication
(10) Pub. No.: US 2009/0081345 A1
(43) Pub. Date: Mar. 26, 2009

(54) Identification of Prion Proteins in Milk

(75) Inventors: Ralph Zahn, Zurich (CH); Nicola Franscini, Zurich (CH); Ahmed El Gedaily, Zurich (CH)

Correspondence Address: THE NATH LAW GROUP, 112 South West Street, Alexandria, VA, 22314, US

(73) Assignee: ALICON AG, Zurich (CH)

(21) Appl. No.: 12/226,899

(22) PCT Filed: Apr. 30, 2007
(86) PCT No.: PCT/EP2007/003800

§ 371 Date: Nov. 25, 2008

(30) Foreign Application Priority Data
May 2, 2006 (EP) 06009023.0

(51) Int. Cl. A23C 9/14; G01N 33/53; G01N 33/553; G01N 33/561

(52) U.S. Cl. 426/491; 436/525; 436/516; 436/501

(57) ABSTRACT

The present invention relates to the use of milk or a derivative thereof for identifying prion proteins, preferably PrP^{Sc} prion proteins, in a mammal. The present invention is also directed to a method for identifying prion proteins, preferably PrP^{Sc} prion proteins, in mammals, comprising the step of contacting milk or a derivative thereof with an agent having high affinity and selectivity for prion proteins, preferably for PrP^{Sc} prion proteins. In addition, a further aspect the present invention concerns a method for removing PrP^{C} and/or PrP^{Sc} prion proteins, preferably PrP^{Sc} prion proteins, from milk or a milk derivative wherein milk or a derivative thereof is contacted with sepharose, preferably sepharose comprising divalent immobilized metal ions.

The present invention relates to the use of milk or a derivative thereof for identifying prion proteins, preferably PrP^{Sc} prion proteins, in a mammal. The present invention is also directed to a method for identifying prion proteins, preferably PrP^{Sc} prion proteins, in mammals, comprising the step of contacting milk or a derivative thereof with an agent having high affinity and selectivity for prion proteins, preferably for PrP^{Sc} prion proteins. In addition, a further aspect the present invention concerns a method for removing PrP^{C} and/or PrP^{Sc} prion proteins, preferably PrP^{Sc} prion proteins, from milk or a milk derivative wherein milk or a derivative thereof is contacted with sepharose, preferably sepharose comprising divalent immobilized metal ions.

### BACKGROUND OF THE INVENTION

Native prion protein, referred to as “PrP^{C}” for cellular prion protein, is widely distributed throughout nature and is particularly well conserved in mammals. The conversion of the native PrP^{C} protein to the infectious protein, referred to as “PrP^{Sc}” for scrapie prion protein or as “PrP^{res}” for proteinase K resistant prion protein, is believed to lead to the propagation of various diseases. Examples of prion-associated diseases include, for example, kuru and Creutzfeldt-Jakob disease (CJD) in humans; scrapie in sheep, bovine spongiform encephalopathy (BSE) in cattle, transmissible mink encephalopathy and wasting disease in deer and elk.

BSE is a form of mad cow disease and is transmissible to a wide variety of other mammals including humans. The human form of BSE is referred to as new variant Creutzfeldt-Jakob disease or vCJD. An estimated 40 million people in the United Kingdom ingested BSE-contaminated beef during the mid- to late 1980s. Because the incubation period for the orally transmitted disease may be 20-30 years, the true extent of this disease may not become apparent until after 2010.

In addition to the ingestion of infected beef, there is a potential for the transmission of prion-associated diseases among humans by blood transfusion. Since there are now (two) direct indications of prion transmission by blood transfusions, there is increasing concern about the security of blood products. Also, the infected prions have already been shown to be present on lymphocytes, and there is also evidence indicating that prions are present in the plasma in addition to being cell-associated. Furthermore, animals can become infected with prion-associated diseases by grazing on prion-contaminated soil or by ingesting hay that contains prion-infected hay mites.

Presently, prion PrP^{Sc} proteins are identified in the central nervous system, blood and lymphoid tissue, in particular in spleen, tonsils, Peyer patches and lymph nodes of infected hosts. Chronic inflammatory states are accompanied by local extravasation of B cells and other inflammatory cells which may induce lymphotoxin-dependent maturation of ectopic FDCs (follicular dendritic cells). Consequently, scrapie infection of mice suffering from nephritis, hepatitis or pancreatitis induces unexpected prion deposits at the sites of inflammation (Helkenwalder et al., Science 307, 1107-1110, 2005). Ligios et al., (Nature Medicine, Vol. 11, No. 11, November 2005, p. 1137-1138) have shown an analogous phenomenon in farm animals. They demonstrate the presence of prion proteins in mammary glands of sheep inflicted with mastitis and scrapie at the same time, a location where blood and lymphocyte cells are recruited following inflammation-associated events.

In summary, prion proteins are found in healthy animals in the central nervous system, blood and lymphoid tissue, in particular in spleen, tonsils, Peyer patches and lymph nodes, and also in TSE—(transmissible spongiform encephalopathy)—infected hosts at sites of inflammation, e.g. nephritis, hepatitis, pancreatitis, mastitis, after recruitment of blood and lymphocyte cells. Hence, the present methods for detecting prion proteins require invasive actions for gathering sample material.

Milk contributes 13% to the worldwide protein supply for humans. The world milk production ranges around 500 million tons per year. On an average a “Swiss brown cow” produces 6,500 litres of milk per year (Swiss “Braunvieh” breeding association, Zug, Switzerland). Before fresh milk reaches the consumer it is usually homogenized and heated. The homogenization procedure involves reducing fat particle size in order to increase consumer tolerance. Heating prolongs the shelf time and inactivates existent pathogens. During pasteurization milk is heated between 72° C. and 75° C. for no more than 30 seconds and immediately cooled down to 4° C. The pasteurized milk is stable for about five days and may contain vitamin and flavor additives. UHT—(ultra high temperature-heated) milk is heated between 1 and 4 seconds to temperatures between 135° C. and 150° C. This procedure kills all conventional pathogens and the milk is fit for consumption for several weeks or months but also contains a reduced amount of nutrients.

Over the last 10 years scientific groups, risk assessment agencies and public health organizations (EC. Scientific Veterinary Committee, Report on the risk analysis for colostrum, milk and milk products (document No. VI/8197196 Version J, Final, 1997); EC. Multidisciplinary Scientific Committee, Opinion on the possible risk related to the use of colostrums, milk and products (1997) have debated the TSE-associated risk for milk and milk products. Epidemiological and bioassay data so far available have not provided evidence for milk to harbor any prion proteins. Therefore, it was concluded that milk is unlikely to present any risk of TSE contamination provided that it originates from healthy animals.

It is the object of the present invention to identify prion proteins, in particular prion PrP^{Sc} proteins, without having to apply invasive methods for obtaining sample material. It is a further object to identify prion proteins, in particular prion PrP^{Sc} proteins, from otherwise healthy animals, i.e. animals that do not suffer from inflammatory conditions next to TSE (transmissible spongiform encephalopathies). Another object of the invention relates to the removal of prion proteins from milk or milk derivatives.

### DESCRIPTION OF THE INVENTION

It was surprisingly found that milk and even processed milk products from mammals contain prion proteins, i.e. prion PrP^{Sc} proteins and/or prion PrP^{C} proteins.

This is highly unexpected for those in the art because PrP^{C} and PrP^{Sc} proteins have so far only been detected in fixed cells or the cellular fraction of blood. Their presence in body fluids is marginal at best. Furthermore, the number of cells normally contained in milk is below 100000 per ml and thus extremely low. In addition, milk contains a relatively high amount of lipids (35 mg/ml) which is known to make protein analysis by common biochemical methods demanding.

To produce one liter milk about 400 to 500 liters of blood must pass through the udder of a cow. While it is not desired to be bound by any theory it seems thus possible that the prion proteins found in milk derive from blood or, alternatively, have been secreted from glandular epithelial cells. Cell types that have been identified in milk from healthy cows are mainly macrophages and other leucocytes. However, in assays below demonstrating the presence of prion proteins cells are completely removed by centrifugation (see example 1). Therefore, the recovered prion proteins were most unlikely cell-associated but rather bound to other proteins or lipids resulting in stable molecular complexes. The fact that milk contains full-length PrP^{C} probably comprising the glycolipid anchor indicates that prion proteins in milk were originally cell-bound and do not represent any of the amino-terminal truncation products of PrP^{C} known to be released from normal cells under physiological conditions (Laffont-Proust et al., FEBS Lett. 579, 6333-6337 (2005); Zhao et al., Virus Res. 115 43-55, 2006).

Therefore, in a first aspect the present invention relates to the use of milk or a derivative thereof for identifying prion proteins in a mammal.

The terms milk and milk derivative are meant to encompass natural milk as well as all processed forms of milk such as, e.g. homogenised milk, pasteurized milk, skimmed milk, UHT (ultra-high temperature-treated) milk, butter, etc. and milk products such as yoghurt, cheese, etc. and even highly processed products containing milk such as, e.g. cakes, pudding, etc.

The term “prion protein” relates to any naturally occurring prion PrP^{C} protein and TSE-(transmissible spongiform encephalitis-) related prion PrP^{Sc} protein as well as to their derivatives resulting from the later processing outside the body, for example, processing of the milk sample for analysis purposes or food processing of said protein. Preferably, the term “prion protein derivative” refers to any fragments of prion proteins that comprise at least one or more prion repeat structures, preferably 2 to 5, more preferably 5 prion repeat structures, preferably prion repeat structures that are an octapeptide, pseudooctapeptide, hexapeptide or pseudohexapeptide, more preferably an octapeptide having a sequence selected from the group consisting of PHGGGWGQ (human), PHGGSWGQ (mouse) and PHGGGWSQ (rat), or a pseudooctapeptide, hexapeptide or pseudohexapeptide derived from said sequences.

Exemplary repeat structures are shown below.

Sheep:
PHGGGWGQPHGGGWGQPHGGGWGQPHGGGGWGQ
(4 repeats)
Cattle:
PHGGGWGQPHGGGWGQPHGGGWGQPHGGGWGQPHGGGGWGQ
(5 repeats)

A recent study has shown that in sheep naturally affected by both scrapie and lymphocyte or lymphofollicular mastitis PrP^{Sc} accumulation was present adjacent to milk ducts (Ligios et al., see above). At least in natural occurring sheep scrapie prion replication can occur following a lymphotropic virus infection in the inflamed mammary gland. This study has neither detected PrP^{C}, PrP^{Sc} nor prion infectivity in milk itself. However, because under such inflammatory conditions the total number of immune cells increases in milk it is highly probable that infectious PrP^{Sc} will reach the milk. In this context milk from animals inflicted with mastitis and TSE is probably also responsible for the spread of scrapie from the ewes to their offspring in affected sheep or goat flocks. Moreover, sheep and goat milk probably also constitutes a TSE exposure risk for humans consuming these products.

In a preferred embodiment, the present invention relates to the use of milk or a derivative thereof for identifying prion PrP^{Sc} proteins in a mammal.

It is noted that the identification of prion proteins does not require that the mammalian source suffers from mastitis or any other inflammatory disease. It was surprisingly found that prion proteins can be identified in milk or a derivative thereof of healthy mammals or mammals inflicted with TSE only. In a preferred embodiment, the use of the present invention relates to the use of milk or a derivative thereof for identifying prion proteins, preferably prion PrP^{C} proteins in healthy mammals, more preferably prion PrP^{Sc} proteins in mammals without inflammatory conditions, most preferably without mastitis.

The present invention is not limited to identifying any particular mammalian prion protein or derivative thereof. In a preferred embodiment, the mammal is selected from the group consisting of human, bovine, ovine, mouse, hamster, deer, goat or rat, preferably bovine and ovine.

The use of milk or a milk derivative according to the present invention for identifying prion proteins in a mammal, in particular for identifying prion PrP^{Sc} proteins, is not limited to any particular method for identifying prion proteins as long as the method is sufficiently selective for prion PrP^{Sc} proteins and/or PrP^{C} proteins.

In a further aspect, the present invention is directed to a method for identifying prion proteins in mammals, i.e. prion PrP^{C} proteins and/or prion PrP^{Sc} proteins, comprising the step of contacting milk or a derivative thereof with an agent having high affinity and selectivity for said prion proteins.

Suitable agents are known and commercially available to those of skill in the art. Typically, these agents are polyclonal or monoclonal antibodies or standard derivatives thereof. Suitable antibodies are provided in the accompanying examples. However, suitable agents for practicing the present invention also include binding proteins from non-immunoglobulin domains. Such binding proteins are reviewed in Binz et al., Nature Biotechnology, Vol. 23, No. 10, October 2005, p. 1257-1268.

Hence and in a preferred embodiment, said agent having high affinity and selectivity for prion PrP^{C} and/or prion PrP^{Sc} proteins is selected from the group consisting of polyclonal or monoclonal antibodies or derivatives thereof and/or binding proteins from non-immunoglobulin domains.

In a preferred method according to the invention the method relates to the identification of prion PrP^{Sc} proteins in a mammal and said agent has high affinity and selectivity for prion PrP^{Sc} proteins.

For practicing the method said mammal is preferably selected from the group consisting of human, bovine, ovine, mouse, hamster, deer, goat or rat.

Because the amount of prion proteins in milk or a milk derivative is very small it may be of advantage in some cases to pretreat the milk or derivative in order to enrich, i.e. concentrate and/or purify the prion proteins.

In a preferred embodiment, the method according to the invention comprises the steps of:
(i) concentrating and/or purifying prion PrP^{C} proteins and/or prion PrP^{Sc} proteins from milk or a derivative thereof,
(ii) contacting milk or a derivative thereof with an agent having high affinity and selectivity for prion PrP^{C} proteins and/or prion PrP^{Sc} proteins.

The term “concentrating and/or purifying” as used herein is meant to indicate that the concentration of prion proteins is raised and/or non-prion proteins and/or non-protein material(s) are removed.

It is noted that the particular technology and methods discussed below based on unligated as well as ligand-modified sepharose relating to the concentration and/or purification of prion PrP^{C} proteins and/or prion PrP^{Sc} proteins, the separation and/or enrichment of prion PrP^{Sc} proteins from PrP^{C} proteins, and the removal of PrP^{C} and/or PrP^{Sc} prion proteins is subject-matter of the applicant's copending international patent applications /EP/2005/011565 filed on 28.10.2005 and PCT/EP2006/010272 filed on 25.10.2006. For practicing the methods of the present invention these particular methods represent preferred embodiments. Other suitable concentrating/purifying, separating and removing methods may be employed, too, for practicing the method of the present invention. However, the methods identified below are considered the best mode for practicing the subject-matter of the invention.

It was surprisingly found that sepharose by itself (i.e. as such, naked, with inactivated, removed, masked ligands) has a specific and high binding affinity to PrP^{Sc} proteins and/or functional derivatives thereof. Therefore, the binding of sepharose to PrP^{Sc} proteins and/or functional derivatives thereof is sufficient for their concentration and/or purification. One merely has to remove the unbound non-prion proteins from said sepharose.

Therefore, in a preferred method according to the invention step (i) comprises the following steps:
a) contacting prion PrP^{C} proteins and/or prion PrP^{Sc} proteins from milk or a derivative thereof with sepharose under conditions that allow for the, preferably specific and high affinity, binding of said sepharose to said prion PrP^{C} proteins and/or prion PrP^{Sc} proteins (preferably prion PrP^{Sc} proteins only),
b) removing the unbound non-prion proteins from said sepharose.
   wherein the sepharose is preferably not a Cu²⁺-chelating sepharose.

If the sepharose is unligated, it will bind to PrP^{Sc} proteins only leaving PrP^{C} proteins unbound. Surprisingly, the sepharose for use in the preferred method of the present invention is not limited to any particular type of sepharose except that the sepharose core should be sufficiently accessible to the prion PrP^{Sc} proteins and/or functional derivatives thereof for binding.

It is preferred that in step a) the sepharose binds with specific and high affinity to prion PrP^{Sc} proteins only and not to prion PrP^{C} proteins, in particular, when detection of prion PrP^{Sc} proteins only is desired.

The term “specific and high affinity binding of sepharose to prion PrP^{Sc}” as used herein is meant to indicate that the sepharose as such (i.e. the sepharose core but not any ligands thereon) binds specifically to PrP^{Sc} but not to PrP^{C}. Preferably, specific binding of sepharose in the context of the invention means the binding of sepharose as such to PrP^{Sc} multimers but not to PrP^{C}. The term high affinity binding in this respect is meant to refer to a binding affinity relating to a dissociation constant of 10⁻⁶ to 10⁻¹² M or lower, preferably 10⁻⁸ to 10⁻¹² M or lower. The skilled person can easily determine a specific and high binding affinity of a given sepharose to prion PrP^{Sc} by routine and simple binding assays. For example, one such assay would comprise the following steps:
a) providing the sepharose to be assayed and removing, inactivating and/or masking any ligands on said sepharose core if present,
b) diluting the PrP^{Sc} used to a concentration that will avoid unspecific removal, e.g. precipitation, unspecific binding, etc.,
c) incubating the sepharose of a) and PrP^{Sc} of b) in a suitable buffer under conditions and for a time that will allow for binding to each other,
d) one or more washing step(s), preferably 3 to 10 buffer volumes incubation buffer, for washing out any unbound protein from the sepharose,
e) optionally washing with an excess, preferably a 1000 fold excess, of unspecifically binding protein, preferably BSA (bovine serum albumin), in order to remove or block any unspecific binding sites on the sepharose,
f) an elution step with a buffer comprising a chaotropic agent, preferably urea and/or guanidinium chloride and/or SDS, in order to remove sepharose-bound PrP^{Sc},
g) detecting PrP^{Sc} in the eluted buffer and, thereby demonstrating high affinity binding of the sepharose to PrP^{Sc} as such.

For determining the specificity of the assayed sepharose, the above assay is repeated except that PrP^{C} instead of PrP^{Sc} is incubated in step c) and PrP^{C} is detected in the wash solution, thereby indicating the lack of binding. Alternatively, PrP^{Sc} and PrP^{C} can be incubated simultaneously with the sepharose in step c) and a specific and high affinity sepharose will result in detecting PrP^{C} in the wash solution and PrP^{Sc} in the chaotropic elution buffer only.

In short, the term “specific and high affinity binding of sepharose to PrP^{Sc} proteins” is meant to distinguish sepharoses and methods using these from sepharoses and said methods that merely bind PrP^{Sc} unspecifically and with low affinity, e.g. by precipitation and/or low adsorption.

The terms “concentrating and/or purifying” as used herein are meant to indicate that the concentration of PrP^{Sc} proteins and/or functional derivatives thereof is raised and/or non-PrP^{Sc} proteins and/or non-protein material(s) are removed.

Preferably, the sepharose is selected from unligated sepharoses, preferably selected from the group consisting of Sepharose 2B®, 4B®, 6B®, Sepharose CL-4B®, Sepharose-6B®, Superdex 75®, Sephacryl 100HR® and Sephadex G10®.

Optionally, further ligands present on the sepharose may be of advantage, for example, when it is desired to bind PrP^{C}, too, or when it is desired to enhance the binding of the sepharose to PrP^{C} and/or PrP^{Sc}.

In a more preferred embodiment, the sepharose is selected from ligand-modified sepharoses, preferably those selected from the group consisting of metal-chelating sepharoses, lectin agaroses, iminodiacetic sepharose, protein A agarose, streptavidin sepharose, sulfopropyl sepharose and carboxmethyl sepharose.

For practicing the preferred methods of the present invention it is necessary that the optional ligands do not mask the sepharose core so that prion PrP^{Sc} proteins and/or functional derivatives thereof have free access. This is the problem with many ligand-modified sepharoses employed in the prior art. The skilled person can routinely select ligand-modified sepharoses that are sufficiently accessible for PrP^{Sc} binding by simply testing the sepharose binding affinity to PrP^{Sc} proteins, and, if desired, design appropriate ligand-modified sepharoses, e.g. by employing spacer molecules that position the ligand at an appropriate distance for the sepharose not to be masked by the ligand.

When identifying prion proteins, it can also be desired or of advantage to separate and/or enrich prion PrP^{Sc} proteins from PrP^{C} proteins.

Another unexpected advantage of the preferred method of the present invention is that the sepharose binding to prion PrP^{Sc} proteins is highly selective with respect to prion PrP^{C} proteins which do not have any significant binding affinity to sepharose by themselves.

When ligand-modified sepharoses are used, wherein the ligand part binds to prion PrP^{C} proteins and/or functional derivatives thereof, the method of the present invention allows for the simultaneous concentrating and/or purification of prion PrP^{Sc} and PrP^{C} proteins. The prion PrP^{Sc} and PrP^{C} proteins can then be separated by selectively removing PrP^{C} proteins and/or functional derivatives thereof from the sepharose.

Hence, in a most preferred embodiment, the method of the invention additionally comprises the step of separating and/or enriching prion PrP^{Sc} proteins from PrP^{C} proteins.

The step of separating and/or enriching prion PrP^{Sc} proteins from PrP^{C} proteins preferably comprises the following additional steps:
a) contacting prion PrP^{Sc} proteins and PrP^{C} proteins from milk or a functional derivative thereof with ligand-modified sepharose under conditions that allow for
   (i) the binding of said sepharose part to said prion PrP^{Sc} proteins, and
   (ii) the binding of said ligand part of the sepharose to PrP^{C} proteins,
b) optionally removing unbound material from said ligand-modified sepharose,
c) optionally waiting for a sufficient time period for some or most of the ligand-bound PrP^{C} proteins and/or functional derivatives thereof to convert into prion PrP^{Sc} proteins and/or functional derivatives in the close proximity of the prion PrP^{Sc} proteins and/or functional derivatives thereof,
d) adding a selective release agent to the sepharose-bound proteins and/or functional derivatives thereof from step a), b) or c) under conditions that allow for the release of PrP^{C} proteins and optionally non-prion proteins from the ligand part of the sepharose but not for the release of the prion PrP^{Sc} proteins and/or functional derivatives thereof from the sepharose part, and
e) removing the PrP^{C} and optionally non-prion proteins from the sepharose.

When prion PrP^{Sc} and PrP^{C} proteins were present on the ligand-modified sepharose it was unexpectedly found that the amount of PrP^{Sc} is raised in many instances at the expense of PrP^{C}. It is believed that PrP^{C} proteins are converted by a spontaneous conformational change in the close proximity of PrP^{Sc} that seem to chaperone this change. This finding is in line with the understanding that the presence of PrP^{Sc} is required for PrP^{Sc} “production” from PrP^{C} precursors.

Moreover, it is preferred that the above method further comprises the step of:
f) releasing PrP^{Sc} prion proteins and/or derivatives thereof from the sepharose.

For releasing PrP^{Sc} prion proteins and/or derivatives thereof from the sepharose it is preferred to add chaotropic agents and/or detergents, preferably urea and/or guanidinium chloride and/or SDS, more preferred to add urea and/or SDS, most preferred to add a gel-loading buffer comprising 8 M urea and 5% SDS and applying an electrical field. Of course, any other non-destructive method routinely applied for interrupting enzymes' affinity to polymers, preferably sugar-derived polymers, can also be employed.

Metal-chelating sepharoses as well as negatively charged sepharoses such as sulfopropyl sepharose and carboxymethyl sepharose may bind to PrP^{Sc} as well as PrP^{C} proteins due to the binding of the sepharose part and optionally the negative charged and/or metal ligand part of the sepharose to PrP^{Sc} and the negatively charged and/or metal ligand part of the sepharose to PrP^{C}.

The mechanism underlying the preferred separation method of the present invention relies on the different binding properties of PrP^{Sc} and PrP^{C} regarding sepharose-immobilized metal ions. While PrP^{Sc} seems to have an intrinsic affinity to sepharose, divalent metal ions and negative charges, PrP^{C} seems to have an intrinsic affinity to divalent metal ions and negative charges only. Hence, their different affinity for sepharose can be employed for separating them.

Preferably, the metal ions of the metal-chelating sepharose are selected from the group consisting Ni²⁺, Zn²⁺, Co²⁺, Mg²⁺, Ca²⁺ and Mn²⁺.

The binding of Ca²⁺ and Mn²⁺ is weaker and both ions bind only monomers of PrP^{Sc} and PrP^{C}.

The other mentioned metal ions Ni²⁺, Co²⁺, Zn²⁺ and Mn²⁺ bind stronger to monomers and oligomers of PrP^{Sc} and PrP^{C} and are preferred for that reason. Because of its excellent binding properties and due to its lack of toxicity under physiological conditions in vivo Zn²⁺ is most preferred for the metal-chelating sepharose for practicing the preferred methods of the present invention.

Incidentally, Cu-sepharose will not retain PrP^{Sc} proteins efficiently. As demonstrated in example one of the above-mentioned copending patent application the reloading of Ni-High Performance Sepharose with Cu²⁺ results in unspecific binding of large amounts of BSA and is, therefore, not suited for the enrichment of prion proteins in complex protein solutions. It is therefore generally preferred for all methods of the invention that the sepharose is not a Cu²⁺-metal-chelating sepharose.

For enriching, i.e. concentrating and/or purifying prion proteins or separating and/or enriching PrP^{Sc} proteins from PrP^{C} proteins the metal-chelating sepharose is preferably Ni-Sepharose, most preferably Ni Sepharose™ High Performance (code number 17-5268-01, 25 ml, 17-5268-02, 100 ml) from Ge Healthcare (Amersham Biosciences Europe GmbH, Industrienstrasse 30, CH-8112 Otelfingen)- or HisTrap HP Column from same company (code number dependent on volume 17-5247-01, 17-5247-05, 17-5248-01, 17-5248-02, 17-5248-05, or 17-5249-01).

When a metal-chelating sepharose is employed for practicing a preferred method of the present invention the selective release agent is preferably a metal chelating agent, preferably an agent selected from EDTA and/or EGTA, more preferably EDTA.

For separating PrP^{Sc} and PrP^{C} proteins and/or functional derivatives thereof from a metal chelating sepharose in a preferred method of the invention, it is most preferred that the metal is Ni²⁺ or Zn²⁺ and the metal chelating agent is EDTA.

It is also preferred that the conditions in step d) of the preferred method of the present invention for separating PrP^{Sc} and PrP^{C} proteins that allow for the release of PrP^{C} and optionally non-prion proteins from the sepharose-immobilized metal ions comprise the presence of a metal chelating agent in a concentration of 10 to 100 mM, more preferably 20 to 80 mM, most preferably EDTA at a concentration of 40 to 80 mM.

It was also found that the addition of small amounts of chelators such as EDTA and/or EGTA to complex protein fractions in milk or milk derivatives can assist to avoid unspecific binding and therefore assists separation of unspecific material from PrP^{Sc} and/or PrP^{C} proteins. For example, for some derivatives it was found that 20 to 40 mM EDTA reduced unspecific binding effectively. When working with sepharose-immobilized metal ions one must take care that the effects of reducing unspecific binding and releasing PrP^{C} by chelators do not overlap if the release of PrP^{C} is not yet desired. Moreover, depending on the presence and amounts of unspecifcally binding proteins the above preferred concentration ranges will have to be adapted, i.e. increased, to compensate for the presence of unspecific proteins that scavenge the chelators for PrP^{C} release. Such an optimization is within the routine skill of those in the art.

Although sepharose itself is sufficient to bind significant amounts of PrP^{Sc} by itself if unmasked it may be desirable to employ sepharoses with at least one additional ligand for specifically binding prion PrP^{Sc} and/or PrP^{C} proteins, wherein said ligand is bound directly or indirectly, e.g. by means of a spacer molecule, to the sepharose.

In a preferred embodiment the additional ligand is selected from the group consisting of prion proteins, functional derivatives of prion proteins, His-tagged prion proteins, prion protein-binding proteins, prion protein-binding antibodies, and prion-protein specific ligands.

More preferably, the additional ligand is a prion protein and/or a functional derivative thereof, e.g. a prion fragment such as e.g. bovine PrP(25-241), that is directly or indirectly bound, e.g. by a metal chelator, to the sepharose.

The reversible aggregation of prion proteins or derivatives thereof with one or more prion repeat structures that oligomerize with prion proteins at a pH of 6.2 to 7.8 and which may dissociate again at a pH of 4.5 to 5.5 provides highly selective and efficient means for binding, concentrating, purifying and/or removing prion proteins and/or functional derivatives thereof (PCT/EP2004 003 060). For practicing a preferred method of the present invention prion repeat structure(s) may be attached to sepharoses as additional ligands in order to specifically oligomerize with prion proteins and thereby to bind these.

In a more preferred embodiment the additional ligand is a prion protein and/or a functional derivative thereof.

The additional ligand on sepharoses for practicing preferred methods of the present invention may be bound to the sepharose directly or indirectly, and is preferably bound by a spacer moiety in between the sepharose and the ligand itself.

Although the preferred methods of the present invention are not limited to any particular prion proteins or derivatives thereof as sepharose ligand the prion proteins and/or functional derivatives thereof for said purpose are selected from the group consisting of prion proteins from human, bovine, ovine, goat, mouse, hamster, deer, or rat origin and derivatives thereof.

The term “functional derivatives of prion proteins” as it is used in the description and the claims refers to any derivatives of prion proteins, in particular fragments thereof, that comprise at least one or more prion repeat structure(s), preferably 2 to 5, more preferably 5 prion repeat structures.

In a preferred embodiment the functional derivative of a prion protein for use as a sepharose ligand has at least one prion repeat structure(s) that is (are) an octapeptide, pseudooctapeptide, hexapeptide or pseudohexapeptide, more preferably an octapeptide having a sequence selected from the group consisting of PHGGGWGQ (human), PHGGSWGQ (mouse) and PHGGGWSQ (rat), or a pseudooctapeptide derived from said sequences, preferably selected from the group consisting of PHGGGGWSQ (various species), and PHGGGSNWGQ (marsupial), or a hexapeptide having a sequence selected from the group consisting of PHNPGY (chicken), PHNPSY, PHNPGY (turtle) or is a pseudohexapeptide derived from said sequences.

In a more preferred embodiment at least one, preferably each, of the prion repeat structures comprises an N-terminal loop conformation connected to a C-terminal β-turn structure.

Most preferred, the functional derivatives for use as sepharose ligands are also capable of reversible aggregation and/or dissociation, i.e. oligomerisation at a pH of 6.2 to 7.8 and/or dissociation of the oligomer aggregate at a pH of 4.5 to 5.5 in an aqueous fluid environment.

The functional derivatives of prion proteins useful as sepharose ligands for practicing the preferred methods of the present invention may also be characterized in that they bind to unmasked sepharose to a significant extent. A significant extent means that preferably at least 50, more preferably at least 70, even more preferably at least 80, and most preferably at least 90% of the derivatives bind to unmasked sepharose relative to the naturally occurring prion protein from which the derivative is derived. For determining the extent of sepharose binding to prion protein derivatives the sepharose binding may be assessed using, e.g. Sepharose® 4 B (Sigma, product code 4B-200). The parameters for such an assay can be routinely determined by those skilled in the art.

As one of average skill in the art of prion proteins will appreciate, the functional derivatives of prion proteins mentioned herein can be briefly and sufficiently characterized in that they comprise at least one of the above prion repeat structures and are capable of binding unmasked sepharose. For bovine prion proteins or derivatives thereof, the binding of a prion protein to sepharose is assumed to be effected by domain 102-241, corresponding to amino acid residues 90 to 230 in human PrP. Analogous regions in prion proteins and derivatives thereof of other species have similar sepharose binding activity.

In a preferred embodiment the functional derivative for use as sepharose ligand for practicing the preferred methods of the present invention is derived from prion proteins by one or more deletion(s), substitution(s) and/or insertion(s) of amino acid(s) and/or covalent modification(s) of one or more amino acid(s).

In a more preferred embodiment the functional derivative for use as sepharose ligand comprises one or more octapeptide repeat sequences, preferably amino acids 51-90, and/or the C-terminal domain, preferably, amino acids 121-230 of human PrP.

The conditions for contacting the prion PrP^{Sc} proteins with sepharose under conditions that allow for the binding of said sepharose to said prion PrP^{Sc} proteins, and optionally the binding of the ligand part of the ligand-modified sepharose to PrP^{C} proteins, if ligand-modified sepharose is employed, are preferably physiological conditions, more preferably a pH of 5 to 8 and 2 to 39° C., more preferably a pH of about 7 and about 20 to 25° C.

Further conditions for binding sepharose to prion proteins are ionic strength, buffer substances, etc. The person skilled in the art can routinely determine the suitable and optimized conditions for binding sepharose to prion proteins.

If sepharoses with the above-mentioned additional ligands for binding prion proteins by prion protein aggregation are used, naturally, a pH of 6.2 to 7.8 is preferred.

In another preferred embodiment the conditions for contacting sepharose and prion proteins comprise the presence of at least one detergent and/or a cell lysis buffer. That way, cells and/or membrane fractions present in a sample of interest can be treated by a method according to the present invention directly without any prerequisite steps for liberating the prion proteins or functional derivatives thereof and making them accessible.

In a last aspect, the present invention relates to a method for removing PrP^{C} and/or PrP^{Sc} prion proteins from milk or a milk derivative, comprising the step of:
(a) contacting milk or a derivative thereof with sepharose under conditions that allow for the binding of said sepharose to said prion proteins.

Suitable sepharoses for removal are discussed above. For the removal of prion proteins metal-chelating sepharoses are preferred.

The term removing as it is used in the context of the removal of prion proteins refers to standard techniques for separating proteins and sepharose material such as centrifugation, filtration, ultrafiltration, etc.

Said metal ions of suitable metal-chelating sepharoses are preferably selected from the group consisting Ni²⁺, Co²⁺, Zn²⁺, Mg²⁺, Ca²⁺ and Mn²⁺, more preferably from the group consisting Ni²⁺, Co²⁺, Zn²⁺ and Mn²⁺, most preferably Zn²⁺ and Ni²⁺.

In a particularly preferred embodiment the metal-chelating sepharose for prion protein removal is Ni Sepharose™ High Performance (code number 17-5268-01, 25 ml, 17-5268-02, 100 ml) from Ge Healthcare (Amersham Biosciences Europe GmbH, Industrienstrasse 30, CH-8112 Otelfingen)- or HisTrap HP Column from same company (code number dependent on volume 17-5247-01, 17-5247-05, 17-5248-01, 17-5248-02, 17-5248-05, or 17-5249-01).

For removing PrP^{C} and/or PrP^{Sc} prion proteins from milk or a milk derivative about 800 μl of 500 mM EDTA are preferably added to 10 ml milk or a derivative thereof before step (a).

It was surprisingly found that prion proteins can be easily and essentially completely removed from milk, commercial milk products or other milk derivatives. The skilled person can now analyse the presence of prion proteins, remove them if desired and verify the result of the removal from milk and derivatives thereof.

Finally, it is noted that the above technology and methods relating to the concentration and/or purification of prion PrP^{C} proteins and/or prion PrP^{Sc} proteins, the separation and/or enrichment of prion PrP^{Sc} proteins from PrP^{C} proteins, and the removal of PrP^{C} and/or PrP^{Sc} prion proteins is subject-matter of the applicants copending, yet unpublished international patent application /EP/2005/011565 filed on 28.10.2005.

In the following the subject-matter of the invention will be described in more detail in examples/embodiments with reference to figures. None of these is to be considered limiting to the scope of the invention as it is set forth in the description and the appended claims.

### FIGURES

FIG. 1 shows a Western Blot of prion PrP^{C} protein after enrichment from 10 ml milk from non-infected cow, sheep, goat, and human using PrioTrap™ matrix according to example 1.

FIG. 2 shows a Western Blot demonstrating the specific binding of several anti-PrP monoclonal antibodies to milk PrP^{C} according to example 2.

FIG. 3 shows a Western Blot demonstrating the effect of PNGase (N-Glycosidase F) treatment on milk and brain PrP^{C} according to example 3.

FIG. 4 shows a Western Blot demonstrating the highly selective removal of PrP^{C} from milk and a silver-stained gel of the same treated and untreated samples demonstrating no effect of the removal treatment on the remaining milk proteins according to example 4.

FIG. 5 shows a Western blot demonstrating the binding capacity of PrioTrap™ to PrP^{Sc} after spiking of milk with brain homogenate according to example 5.

### EXAMPLES

### Example 1

### Detection of Native PrP^{C} in Milk of Human and Animals

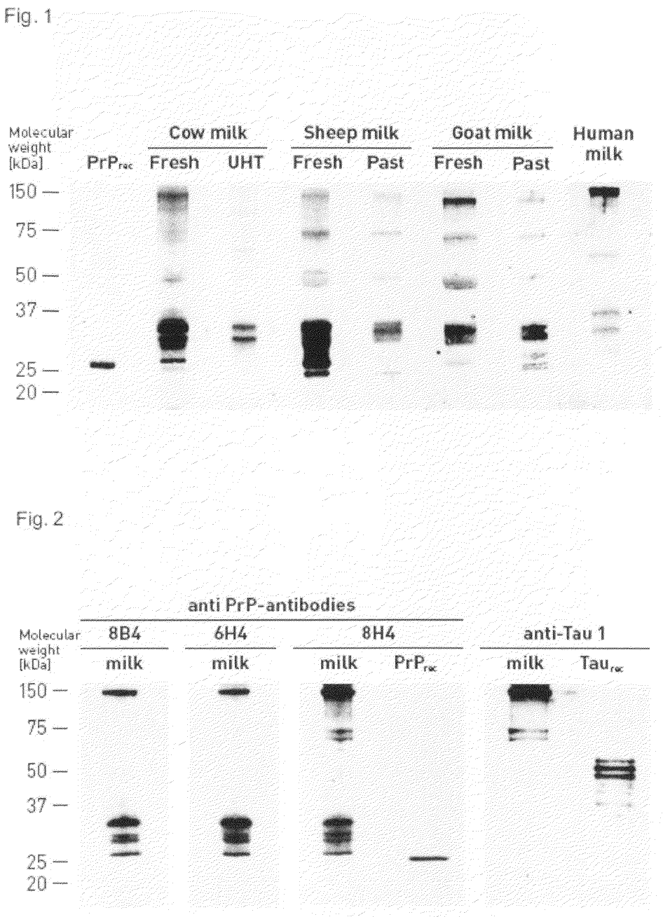

A volume of 10 ml milk (fresh or UHT (standard ultra high temperature treatment) or pasteurised) was centrifuged at 3000 g for 10 min to ensure the complete removal of cells. The cell-free and fat-poor milk supernatant was incubated with 800 μl of 500 mM EDTA solution pH 7.4 and stirred for 30 min in the presence of 50 μl PrioTrap™ matrix (Ni Sepharose™ High Performance (code number 17-5268-01, 25 ml, 17-5268-02, 100 ml) from Ge Healthcare (Amersham Biosciences Europe GmbH, Industrienstrasse 30, CH-8112 Otelfingen). The matrix was washed four times at RT with 10 ml washing solution containing 100 mM sodium phosphate, 20 mM Tris, 10 mM imidazol buffer pH 8. To elute the proteins from the matrix, 15 μl sample buffer (XT sample buffer, Biorad, Biorad Laboratoires Nenzlingerweg 2, 4153 Reinach, CH) were added and heated for 10 min at 70 C.°. The matrix containing sample buffer was loaded on a Criterion XT 12% Precast gel (Biorad). After electrophoreses, proteins were transferred onto a PVDF membrane (Hybond-P, Amersham Biosciences) by a semi-dry transfer, using a three buffers system (anode 1: 300 mM Tris, 20% methanol, pH 10.4; anode 2: 25 mM Tris, 20% methanol, pH 10.4; cathode: 25 mM Tris, 40 mM aminohexanoic acid, 0.05% SDS, pH 9.4). Prion proteins were detected by Western Blotting using the monoclonal antibody PrP-mab 8B4 (alicon AG; Product number A0001; Schlieren, Switzerland; Li et al., J. Mol. Biol. 301, 567-573, 2000)) in combination with ECL Advance Western Blotting Detection Kit, Amersham Biosciences. The molecular weight markers are indicated on the left side. Recombinant bovine PrP (aa 25-241) was used as a standard. The results are shown in FIG. 1, a Western Blot of PrP^{C} after enrichment from 10 ml milk from non-infected cow, sheep, goat, and human using PrioTrap™ matrix (see above). In cow milk three PrP^{C} isoforms are observed with an apparent molecular mass of about 34 kD, 30 kD, and 27 kD corresponding to diglycosylated, monoglycosylated, and unglycosylated PrP^{C}, respectively. Furthermore, in some preparations monoglycosylated PrP^{C} appears as a double band, indicating that the two glycosylation sites may be linked to different carbohydrates. The apparent molecular mass of unglycosylated PrP^{C} is slightly higher when compared to a recombinant bovine PrP(25-241) standard at 26 kD, indicating that native PrP^{C} in milk contains a glycosyl phosphatidylinositol anchor (Stahl et al, Cell, 51, 229-240, 1987; Stahl et al., Biochemistry 29, 8879-8884, 1990). About the same distribution of PrP^{C} isoforms is observed for sheep, goat, and human milk, although the total amount of native PrP^{C} significantly differs between the species. The relative ratio of sheep/cow/goat/human PrP^{C} is estimated at 100/20/4/1.

From experiments performed on sequential incubations with PrioTrap™ the total concentration of PrP^{C} in fresh cow milk can be estimated to be about 200 pg/ml. Taking into account the relative ratios of PrP^{C} in milk of different species, fresh sheep milk and goat milk contain about 1 ng/ml and 40 pg/ml PrP, respectively. Human breast milk contains less than 10 pg/ml PrP^{C}. The concentration of PrP^{C} in Swiss off-the-shelf milk is reduced when compared to fresh milk but can clearly be detected (FIG. 1). About the same concentration of PrP^{C} was measured for organic farm milk and non-organic farm milk as well as for pasteurized and ultra-high temperature (UHT) treated milk when compared from the same supplier (data not shown).

### Example 2

### Specific Binding of Anti-PrP Monoclonal Antibodies to Milk PrP^{C}

To confirm the specificity of the immunochemical detection of PrP^{C} in milk different anti-PrP monoclonal antibodies were compared which are directed against non-overlapping epitopes (FIG. 2).

Fresh cow milk samples were treated as described for example 1 except that various first antibodies were used for the detection of PrP^{C} in fresh cow milk: PrP-mab 8B4 (alicon AG, see above), mAB 6H4 (Prionics AG, Switzerland; Korth et al. Nature, 390, 74-77, 1997), and PrP-mab 8H4 (alicon AG, Product number A0002; Schlieren, Switzerland; Zanusso et al., Proc. Natl. Acad. Sci. USA 95, 8812-8816, 1998). A tau-1 protein-specific monoclonal antibody (Chemicon International, Inc., California USA) was used as a negative control. PrP-mab 8B4 binds to residues 37-44 within the flexibly disordered amino-terminal domain of mouse PrP; mAB 6H4 targets residues 144-152 within helix 1 of the globular carboxy-terminal domain; and PrP-mab 8H4 binds to residues 175-185 of helix 2 within the globular domain. The three antibodies recognize the same proteins and thus confirm the presence of PrP^{C} in milk. In control experiments, with a non-PrP antibody, e.g., anti-Tau protein monoclonal antibody (FIG. 2) and anti-AB monoclonal antibody (Calbiochem, Germany; data not shown), none of the PrP^{C} isoforms was detected, thus confirming binding specificity of the anti-PrP monoclonal antibodies. An interesting observation with regard to antibody 8B4 is its “clear” detection profile when compared to 6H4 and 8B4 antibodies. This can be rationalized by 8B4 not recognizing a variety of carboxy-terminal fragments of milk PrP^{C} which appear as smear in the Western Blot.

### Example 3

### Identification of PrP-Glycoforms by PNGase Treatment of Milk and Brain PrP^{C}

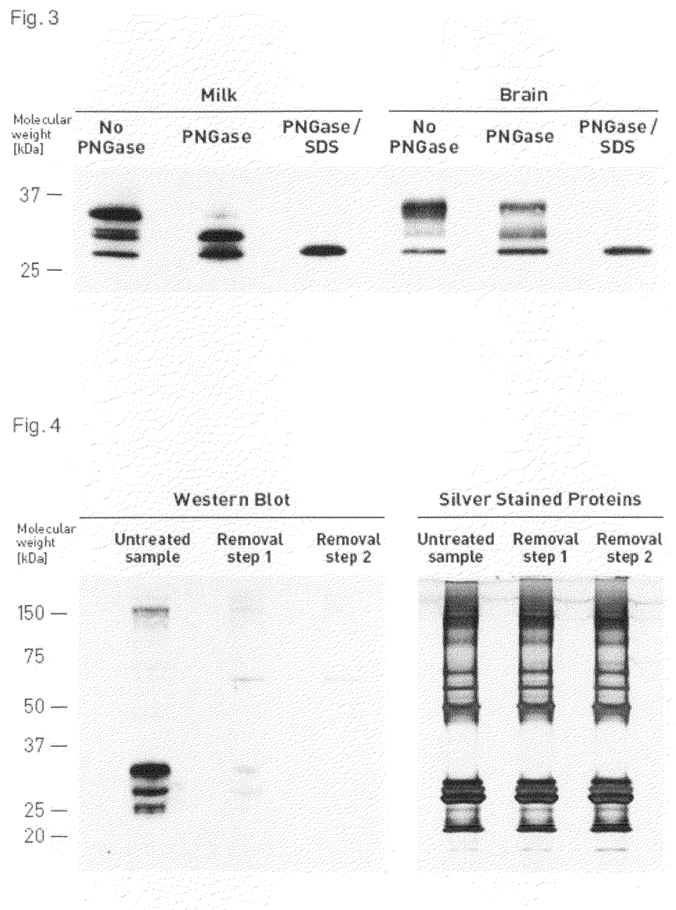

Identification of PrP-glycoforms was performed with PNGase (FIG. 3), an enzyme that cuts off oligosaccharides from N-linked glycoproteins, e.g., the two N-linked sugars of PrP^{C} (Haraguchi et al., Arch. Biochem. Biophys. 274, 1-13, 1989).

For PNGase treatment prion protein extracted from 10 ml fresh cow milk as described in example 1 or 10 μl of 1% (w/v) cow brain homogenate was incubated under shaking for 12 h at 37 C.° in buffer containing 100 mM sodium phosphate, 10 mM Tris, 20 mM Imidazol 1% NP-40, 1% MEGA-8, pH 8, and 1.5 units of N-Glycosidase F (Roche, Mannheim, Germany). Under more stringent cleavage conditions, proteins were denatured by heating for 10 minutes at 100° C. in the presence of 0.5% SDS before treatment with 4 units of N-Glycosidase. Proteins were analyzed by SDS polyacrylamide gel electrophoresis and Western Blotting using PrP-mab 8B4 antibody as described in example 1. After partial cleavage with PNGase the upper PrP-isoform in the Western Blot representing diglycolysated PrP^{C} (34 kD) disappears in favour of monoglycosylated (30 kD) and nonglycosylated PrP^{C} (27 kD). In parallel, there seems to be a small shift from the higher molecular weight monoglycosylated form to the lower molecular weight form. A slight downshift of the monoglycosylated PrP^{C} is also observed for brain homogenate after PNGase treatment (FIG. 3). The diglycosylated PrP^{C} isoforms differ slightly in molecular mass, indicating that carbohydrate structure of PrP^{C} in milk and brain may not be identical. More stringent reaction conditions result in complete truncation of carbohydrates from PrP^{C}. Most importantly, the apparent molecular masses of non-glycosylated PrP^{C} in milk exactly matches with that of the corresponding PrP^{C} in brain homogenate.

### Example 4

### Removal of PrP^{C} from Milk

PrioTrap™ can also be applied for removing PrP^{C} from milk. PrP^{C} was detected in 10 ml fresh cow milk as described above in example 1. Between detection steps the milk was incubated with 500 μl PrioTrap™ (Ni-Sepharose™ High Performance GeHealthcare, Amersham Biosiences) for 30 min to remove PrP^{C}. Supernatants before and after the removal steps were analyzed by Western Blotting as described in example 1 and total milk proteins were detected by silver staining (SilverSNAP Stain Kit II, Pierce, Perbioscience, Lausanne, Switzerland).

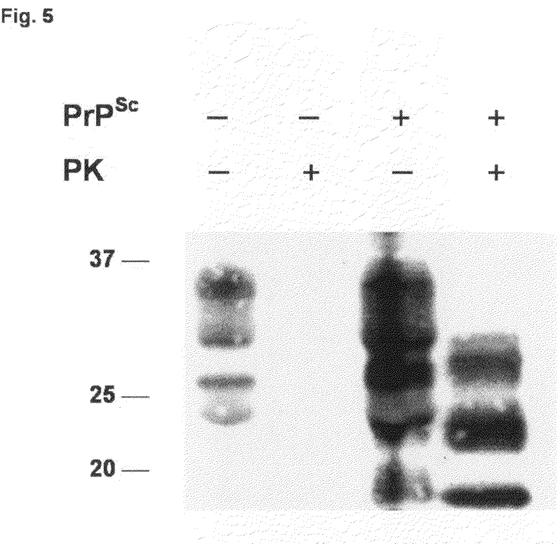

As shown in FIG. 4, after the first treatment of 10 ml milk with matrix more than 95% of endogenous PrP^{C} was already removed, and after the second treatment PrP^{C} was completely removed within the detection limit of 1 pg. However, the overall protein concentration (measured by bicinchoninic acid assay, Perbioscience Switzerland) was constant with about 40 mg/ml before and after PrP^{C} elimination. The protein composition of milk as analyzed by SDS PAGE (FIG. 4B) was not affected either. Prion protein was also completely removed when milk was spiked with PrP^{Sc} from mouse Rocky Mountain Laboratory (RML) brain homogenate (data not shown). Thus, PrioTrap™ can be used for enrichment and identification of prion proteins in milk and, also, for its complete removal.

### Example 5

### Detection of PrP^{Sc} in Milk

PrioTrap™ can also be applied for the detection of PrP^{Sc} in milk. 10 ml pasteurized sheep milk were spiked with 10 μl 1% (w/v) mouse Roky Mountain Laboratory (RML) brain homogenate containing about 1 ng PrP^{Sc}. After stirring for 30 min milk was centrifuged at 3000 g for 10 min. The pellet fraction was dissolved in 10 ml 100 mM sodium phosphate, 20 mM Tris, 10 mM imidazol buffer, pH8 containing 1% triton-X 100 and subsequently incubated under shaking for 30 min in the presence of 50 μl PrioTrap™. Subsequently, the matrix was washed four times with 10 ml washing solution containing 100 mM sodium phosphate, 20 mM Tris, 10 mM imidazol buffer pH 8. Matrix bound proteins were treated with 0.88 U proteinase K (PK) (Roche, Switzerland) for 60 min at 37 C.°. The reaction was stopped by adding PMSF to a final concentration of 3 mM. The proteins were analysed by Western blotting as described in example 1 with 8H4 antibody.

The three downshifted bands detected after PK treatment in the milk spiked with brain homogenate clearly represent PrP^{Sc} after enrichment with PrioTrap™ (see FIG. 5). The

1. Use of milk or a derivative thereof for identifying prion proteins in a mammal.

2. The use according to claim 1, wherein the prion proteins are prion PrP^{Sc} proteins.

3. The use according to claim 1, wherein the mammal is selected from the group consisting of human, bovine, ovine, mouse, hamster, deer, goat or rat.

4. A method for identifying prion proteins in mammals, comprising the step of:
contacting milk or a derivative thereof with an agent having high affinity and selectivity for prion proteins.

5. The method of claim 4, wherein the prion proteins are prion PrP^{Sc} proteins and the agent has high affinity and selectivity for PrP^{Sc} prion proteins.

6. The method of claim 4, wherein the mammal is selected from the group consisting of human, bovine, ovine, mouse, hamster, deer, goat or rat.

7. The method of claim 4, wherein the agent having high affinity and selectivity for prion PrP^{C} and/or prion PrP^{Sc} proteins is selected from the group consisting of polyclonal or monoclonal antibodies or derivatives thereof and/or binding proteins from non-immunoglobulin domains.

8. The method of claim 4, comprising the steps of:
(i) concentrating and/or purifying prion PrP^{C} proteins and/or prion PrP^{Sc} proteins from milk or a derivative thereof,
(ii) contacting milk or a derivative thereof with an agent having high affinity and selectivity for prion PrP^{C} proteins and/or prion PrP^{Sc} proteins.

9. The method according to claim 8, wherein step (i) comprises the following steps:
a) contacting prion PrP^{C} proteins and/or prion PrP^{Sc} proteins from milk or a derivative thereof with sepharose under conditions that allow for the binding of said sepharose to said prion PrP^{C} proteins and/or prion PrP^{Sc} proteins,
b) removing the unbound non-prion proteins from said sepharose.

10. The method according to claim 9, wherein the sepharose is selected from unligated sepharoses, preferably selected from the group consisting of Sepharose 2B®, 4B®, 6B®, Sepharose CL-4B®, Sepharose-6B®, Superdex 75®, Sephacryl 100HR® and Sephadex G10®.

11. The method according to claim 9, wherein the sepharose is selected from ligand-modified sepharoses, preferably selected from the group consisting of metal-chelating sepharoses, lectin agaroses, iminodiacetic sepharose, protein A agarose, streptavidin sepharose, sulfopropyl sepharose and carboxmethyl sepharose.

12. The method according to claim 8, additionally comprising the step of separating and/or enriching prion PrP^{Sc} proteins from PrP^{C} proteins.

13. The method according to claim 12, comprising the following additional steps:
a) contacting prion PrP^{Sc} proteins and PrP^{C} proteins from milk or functional derivatives thereof with ligand-modified sepharose under conditions that allow for
(i) the binding of said sepharose part to said prion PrP^{Sc} proteins, and
(ii) the binding of said ligand part of the sepharose to PrP^{C} proteins,
b) optionally removing unbound material from said ligand-modified sepharose,
c) optionally waiting for a sufficient time period for some or most of the ligand-bound PrP^{C} proteins and/or functional derivatives thereof to convert into prion PrP^{Sc} proteins and/or functional derivatives in the close proximity of the prion PrP^{Sc} proteins and/or functional derivatives thereof,
d) adding a selective release agent to the sepharose-bound proteins and/or functional derivatives thereof from step a), b) or c) under conditions that allow for the release of PrP^{C} proteins and optionally non-prion proteins from the ligand part of the sepharose but not for the release of the prion PrP^{Sc} proteins and/or functional derivatives thereof from the sepharose part, and
e) removing the PrP^{C} and optionally non-prion proteins from the sepharose.

14. The method of claim 13, further comprising the step of:
f) releasing PrP^{Sc} prion proteins and/or derivatives thereof from the sepharose.

15. The method of claim 14, wherein the release of PrP^{Sc} prion proteins and/or derivatives thereof is accomplished by adding chaotropic agents and/or detergents, preferably urea and/or guanidinium chloride and/or SDS, more preferably adding urea and/or SDS, most preferably adding a gel-loading buffer comprising 8 M urea and 5% SDS and applying an electrical field.

16. The method of claim 11, wherein the ligand-modified sepharose is a metal-chelating sepharose comprising divalent immobilized metal ions.

17. The method of claim 16, wherein the metal ions are selected from the group consisting Ni²⁺, Co²⁺, Zn²⁺, Mg²⁺, Ca²⁺ and Mn²⁺.

18. The method of claim 17, wherein the metal ions are selected from the group consisting Ni²⁺, Co²⁺, Zn²⁺ and Mn²⁺, preferably Ni²⁺ and Zn²⁺.

19. The method of claim 18, wherein the ligand-modified sepharose is Ni Sepharose™ High Performance from GeHealthcare, Amersham.

20. The method of any one of claims 13, wherein the ligand-modified sepharose is a metal-chelating sepharose comprising divalent immobilized metal ions and the selective release agent is a metal chelating agent, preferably an agent selected from EDTA and/or EGTA.

21. The method of claim 20, wherein the metal chelating agent is EDTA.

22. The method according to claim 21, wherein the metal chelating sepharose comprises Zn²⁺ and the metal chelating agent is EDTA.

23. The method according to claim 13, wherein the conditions in step d) that allow for the release of non-prion proteins and PrP^{C} from the sepharose-immobilized metal ions comprise the presence of a metal chelating agent in a concentration of 10 to 100 mM, more preferably 20 to 80 mM, most preferably EDTA at a concentration of 40 to 80 mM.

24. The method of claim 9, wherein at least one additional ligand for binding prion PrP^{Sc} and/or PrP^{C} proteins is bound directly or indirectly to the sepharose.

25. The method of claim 24, wherein the additional ligand is selected from the group consisting of prion proteins, functional derivatives of prion proteins, His-tagged prion proteins, prion protein-binding proteins, prion protein-binding antibodies, and prion-protein specific ligands.

26. The method of claim 25, wherein the additional ligand is a prion protein and/or a functional derivative thereof.

27. The method of claim 17, wherein the additional ligand is bound to sepharose directly or indirectly, preferably by a spacer moiety.

28. The method of claim 9, wherein the conditions for the binding of sepharose to prion PrP^{Sc} proteins are physiological conditions, preferably a pH of 5 to 8 and 2 to 39° C., more preferably a pH of about 7 and about 2 to 8° C.

29. The method of claim 28, wherein the conditions comprise the presence of at least one detergent and/or a cell lysis buffer.

30. A method for removing PrP^{C} and/or PrP^{Sc} prion proteins from milk or a milk derivative, comprising the step of:
(a) contacting milk or a derivative thereof with sepharose under conditions that allow for the binding of said sepharose to said prion proteins,
b) removing the milk or milk derivative from said sepharose.

31. A method according to claim 30, wherein said sepharose is a metal-chelating sepharose comprising divalent immobilized metal ions.

32. The method of claim 31, wherein the metal ions are selected from the group consisting Ni²⁺, Co²⁺, Zn²⁺, Mg²⁺, Ca²⁺ and Mn²⁺, preferably from the group consisting Ni²⁺, Co²⁺, Zn²⁺ and Mn²⁺, more preferably, wherein the metal ions are Zn²⁺.

33. The method according to claim 31, wherein the metal-chelating sepharose is Ni Sepharose™ High Performance from GeHealthcare, Amersham.

34. The method according to claim 30, wherein about 800 μl of 500 mM EDTA are added to 10 ml milk or a derivative thereof before step (a).
